(19) United States
(12) Patent Application Publication
(10) Pub. No.: US 2024/0277757 A1
(43) Pub. Date: Aug. 22, 2024

(54) METHODS OF USING A PLASMA-GENERATED STREAM OF NO-CONTAINING GAS FOR TREATMENT OF A SPECTRUM OF MEDICAL CONDITIONS

(71) Applicant: Origin Life Sciences, Inc., Princeton, NJ (US)

(72) Inventors: Alexander DOLGOPOLSKY, Princeton, NJ (US); Victor N. VASILETS, Princeton, NJ (US); Michael D. PRESTON, Princeton, NJ (US); Kirill GUTSOL, Princeton, NJ (US); Howard NELSON, Princeton, NJ (US)

(73) Assignee: Origin Life Sciences, Inc., Princeton, NJ (US)

(21) Appl. No.: 18/570,363

(22) PCT Filed: May 26, 2022
(86) PCT No.: PCT/US2022/031027

§ 371 Date: Dec. 14, 2023

Related U.S. Application Data
(60) Provisional application No. 63/211,726, filed on Jun. 17, 2021.

(51) Int. Cl. A61K 33/00; A61K 9/00

(52) U.S. Cl. CPC A61K 33/00; A61K 9/0014

(57) ABSTRACT

Methods for administering nitric oxide (NO) generated by a plasma source via stream of gas also containing a number of other chemical and physical components that create synergistic effect in treatment of a variety of medical conditions. A stream of gas is generated by a plasma source/sources, in which nitric oxide is a part of its content and in desired concentration and at the appropriate speed, so that therapeutically significant amount of NO is delivered per unit of treated tissue area and per unit of time. The stream also contains other physical and chemical compounds such as H₂O₂, OH, O₂, O₂⁻ (i.e., oxygen superoxide), heating and UV radiation. The stream of gas generated by plasma source(s) must be precisely directed to the site of action to achieve the best therapeutic result.

### FIELD OF THE DISCLOSURE

The present disclosure relates in general to the field of medical treatment and, more particularly, to methods for effectively administering a stream of gases generated by arc plasma discharge and containing nitric oxide (NO) together with other molecules, heating and radiation acting synergistically with nitric oxide in the treatment of medical conditions and diseases. The present disclosure provides that in spite of the extreme importance of administering nitric oxide (NO) to the site of treatment, the effect of other components present in the plasma-generated stream of gas cannot be trivialized. The disclosure provides an extensive analysis of at least one embodiment of the plasma device creating NO-rich stream of gas using mass spectrum and speckled Doppler technologies. Treatment protocols for a number of particular medical conditions using the said embodiment of the device are also provided.

### BACKGROUND OF THE DISCLOSURE

Nitric Oxide (NO) gas is a short-lived molecule normally found in a gaseous state both inside and outside the human body. NO is a signaling molecule known to have numerous regulatory, protective, and therapeutic properties. Augmenting the body's natural generation of NO by either stimulating increased production of endogenous NO or introducing exogenously-produced NO into the body can improve the body's response to damage, pain, and invading organisms. However, it is difficult to deliver NO into living tissue and, in its gaseous state, NO without a specialized mechanism of delivery does not penetrate through the dermis. To be clinically useful, NO together with other components present in the plasma stream of gases must be delivered to the site of action, and in sufficient quantity per unit area and per unit time.

Prior methods for delivering NO for therapeutic purposes include the administration of chemical compounds which release NO into the body. Other methods employ NO pathway agonists and NO antagonists. Still other methods employ high-pressure NO gas and sprays. Yet another method involves surrounding a body with sealed vacuum containers into which gaseous NO is introduced. Attempts have also been made to force pressurized NO through tissue and skin. For various reasons, these methods have yielded limited results. For example, gaseous NO is highly reactive, has a low diffusion coefficient and has an extremely short lifetime in tissue media.

Another method that has failed to achieve meaningful clinical success involves the administration of molecular donors, which has been demonstrated to be problematic because the control of the release of the payload cannot be modulated, nor can the penetration/saturation of the donors be reliably modulated.

There are several solutions that target specific clinical outcomes involving NO. Sildenafil citrate (sold under the brand name VIAGRA), for example, interferes with the down regulation of NO in erectile dysfunction syndrome. Etanercept (sold under the brand name ENBREL), for example, uses an anti-TNF alpha antibody to do what NO would do in inflammatory diseases of the joint. Most solutions involve affecting the NO pathways, due to the difficulty in stimulating production of NO directly at the site of action. Because of lack of site-specificity of these NO pathways, pharmacological negative side effects can be serious.

### SUMMARY

In view of the forgoing, it would be advantageous to provide a method for administering NO-containing gas together with other chemical and physical compounds such as H₂O₂, OH, O₂, superoxide (O₂⁻), UV radiation and heating, at a site of action in a manner that facilitates therapeutic benefits.

In accordance with the present disclosure, therapeutic methods of administering NO in synergistic combination with other components present in the plasma-generated stream of gases to achieve a therapeutic benefit are disclosed. In some embodiments, the methods include employing the exogenous production and application of NO together with other components present in the plasma-generated stream of gases by high-temperature plasma conversion of air. In other embodiments, the NO together with other components present in the plasma-generated stream of gases is applied to a treatment site to facilitate the repair and growth of living tissue in humans, animals, and plants.

The methods of the present disclosure operate to selectively apply a NO-containing stream of gas together with other stream components to a treatment site for the beneficial effects evident with increased levels of NO and other components of the stream associated with the cellular and tissue environment. The methods more particularly include employing an apparatus capable of producing a gas stream of a desired composition including NO. The apparatus may be used to apply a desired level of NO, via matter, together with other components of the stream, to a treatment site.

### BRIEF DESCRIPTION OF THE DRAWINGS

FIGS. 1-4 illustrates an exemplary plasma arc discharge device for producing NO containing stream of gas according to the present disclosure.

FIG. 5 is a flow diagram illustrating a first exemplary method in accordance with an embodiment of the present disclosure.

FIG. 6 is a graph illustrating the results of mass-spectroscopy analysis illustrating all important compounds of the NO-containing gas stream: N₂, O₂, NO, NO₂, H₂O₂, Ar, and O₃.

FIG. 7 is a graph illustrating a spectral distribution of UV radiation from a plasma arc emitted from the plasma arc discharge device of FIGS. 1-4.

FIG. 8 is a graph illustrating the temperature of the NO-containing plasma stream emitted from the plasma arc discharge device of FIGS. 1-4. The measurements were made in the open-air environment.

FIG. 9 illustrates the plasma arc discharge device of FIGS. 1-4 embodied in an applicator, as developed by Origin, Inc.

FIG. 10 illustrates the plasma arc discharge device of FIGS. 1-4 embodied in an applicator, include a base unit, monitor, and keyboard, as developed by Origin, Inc.

FIG. 11 is a graph illustrating blood flow prolongation effect of NO measured by speckled Doppler measurement device in 6 to 12 min range.

FIG. 12 is a graph illustrating blood flow prolongation effect of NO measured by speckled Doppler measurement device in 6 min range vs. just thermal influence without the application of NO.

### DETAILED DESCRIPTION

In accordance with the present disclosure, a method and apparatus are presented for creating a discrete stream of matter generated by plasma discharge, where the most prominent component of the stream is NO and administering such stream to human tissues to obtain a therapeutic result. In some embodiments, NO application at the surface level (i.e., directed at the skin or open wound) is believed to stimulate the body's own production of NO such that therapeutic effects can be obtained at and around the indication site. Alternatively, the disclosed methods may exploit the fact that the NO-containing plasma stream generated by a plasma source has sufficiently high energy and velocity so that it can penetrate through and around cellular membranes. In some cases, nitric oxide may pass through biofilms and the stratum corneum to produce therapeutic results in the associated tissue.

In one embodiment the NO-containing plasma stream of gas contains a mixture of eight main stable molecules N₂, O₂, Ar, H₂O, NO, H₂O₂, H₂, NO₂ and unstable radicals H, N, O, and OH, which usually stay in the plasma stream after partial quenching. One of the key points of the current disclosure is that of a synergistic effect of NO together with other plasma components such as H₂O₂, O₂, and O₂⁻ at elevated gas temperature (40-60° C.) plus the UV radiation from the plasma source. The synergy of the above components of the gas stream can significantly improve (accelerate) both the antimicrobial action and tissue-regeneration processes (i.e., therapeutic effects) of nitric oxide generated by a plasma source, in comparison with pure chemically-produced NO gas from a compressed gas cylinder.

Calculations based on classical thermodynamics give the following illustration of the key base products in a stream emitted from an exemplary source of plasma:

The synergistic effects of the disclosed NO-containing stream in treating various medical conditions have been supported by the results of numerous publications. Examples are listed below, all of which are incorporated by reference in their entirety:
1. The mixture of NO and H₂O₂ in the plasma gas could substantially increase the antimicrobial effect
   R. Pacelli et al., “Nitric oxide potentiates hydrogen peroxide-induced killing of Escherichia coli.,” Journal of Experimental Medicine, vol. 182, no. 5, pp. 1469-1479 November 1995, doi: 10.1084/jem.182.5.1469
2. The mixture of NO and H₂O₂ is not toxic for mammalian cells because NO could inhibit toxicity of H₂O₂.
   S. Kotamraju et al., “Nitric oxide inhibits H2O2-induced transferrin receptor-dependent apoptosis in endothelial cells: Role of ubiquitin-proteasome pathway,” Proceedings of the National Academy of Sciences, vol. 100, no. 19, pp. 10653-10658, September 2003, doi: 10.1073/pnas.1933581100
   Y. Yoshioka, T. Kitao, T. Kishino, A. Yamamuro, and S. Maeda, “Nitric Oxide Protects Macrophages from Hydrogen Peroxide-Induced Apoptosis by Inducing the Formation of Catalase,” J Immunol, vol. 176, no. 8, pp. 4675-4681 April 2006, doi: 10.4049/immunol.176.8.4675.
3. It is well known in pulmonology that the mixture of NO and O₂, could improve therapeutic regenerative effects.
   Chotigeat U, Khorana M and Kanjanapattanakul W, 2007 Inhaled nitric oxide in newborns with severe hypoxic respiratory failure J. Med. Assoc. Thai. 90 266-71
4. Hydrogen peroxide in small concentrations could improve wound closure.
   Roy S, Khanna S, Nallu K, Hunt T K and Sen C K, 2006 Dermal wound healing is subject to redox control Mol. Ther. 13 211-20 (topical application of low (0.15%) concentrations of H₂O₂ presented in plasma gas mixture stimulates the wound closure process by activating redox-dependent pathways)
5. The mixture of NO and O₂⁻ also present in plasma gas could improve blood coagulation and effect on endothelial-cell relaxation factor:
   Krotz F, Sohn H Y and Pohl U, 2004 Reactive oxygen species-players in the platelet game, Arterioscler Thromb. Vasc. Biol. 24 1988-96
   Clancy R M, Leszczynskapiziak J and Abramson S B, 1992 Nitric oxide, an endothelial-cell relaxation factor, inhibits neutrophil superoxide anion production via a direct action on the NADPH oxidase J. Clin. Invest. 90 1116-21
6. The presence of toxic NO₂ at low concentration (less than 25 ppm) in plasma gas could be dangerous for bacteria but not for mammalian cell.
   Emerging Pollutants-Some Strategies for the Quality Preservation of Our Environment. Chapter 2. S. Depayras, T. Kondakova, H. J. Heipieper, M. Feuilloley, N. Orange and C. Duclairoir-Poc. “The Hidden Face of Nitrogen Oxides Species: From Toxic Effects to Potential Cure?” http://dx.doi.org/10.5772/intechopen.75822
7. Heating of tissue to the temperatures 40-60° C. by plasma gas and UV radiation from plasma could be also considered as synergy factors.

The increase of the temperature causes the acceleration of all chemical and biological processes which are responsible for the Anti-Bacterial Action/Sterilization and Regeneration Processes mentioned above. UV radiation could evidently improve the Anti-Bacterial Action/Sterilization of NO and H₂O₂ plasma components.
R. Abshire Ultraviolet Radiation: A Method of Sterilization in the Pharmaceutical Industry. Ozone: Science & Engineering. 1988. Volume 10, Issue 1, pp. 25-38 https://doi.org/10.1080/01919518808552505

Summarizing the examples above, the differential response of mammalian cells and bacteria to the mixture NO+H₂O₂ in the NO-containing plasma stream creates unique conditions combating invading microorganisms with minimal damage to the host cells. As a result, NO-rich gas, together with other accompanying components generated by the plasma source, in contrast to nitric oxide gas from the tank provides better selectivity in killing bacteria with minimal, or no damage at all, to mammalian cells. This happens due to the synergistic effect of NO with H₂O₂, possibly OH, and UV radiation at 40-60° C. range of temperatures. At the same time, the synergistic effect of NO generated by the plasma source together with other oxygen-containing species (such as O₂, O₂⁻) at an elevated temperature improves tissue-regeneration processes.

The present disclosure illustrates the presence of components with demonstrable synergistic effects mentioned above by mass-spec analysis of at least one stream of NO-containing gas generated by the plasma source.

A gas stream generated by a plasma source that contains NO together with other stream components synergistically acting in treatment procedures can be created via several methods. Atmospheric air contains nitrogen and oxygen and, thus, plasma energy generated in sufficient amount via a favorable geometric arrangement can produce NO from within the gaseous mixture. In one non-limiting, exemplary embodiment, a pre-formed gaseous N₂—O₂ mixture can be created and passed through an arc discharge plasma that supplies sufficient energy to produce NO and other components in a resulting stream.

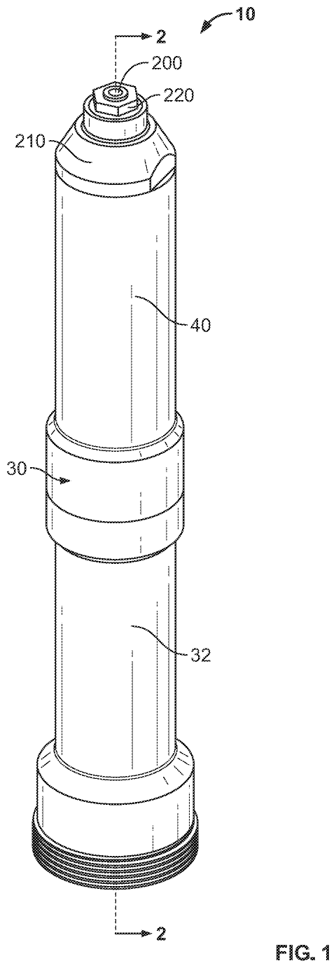

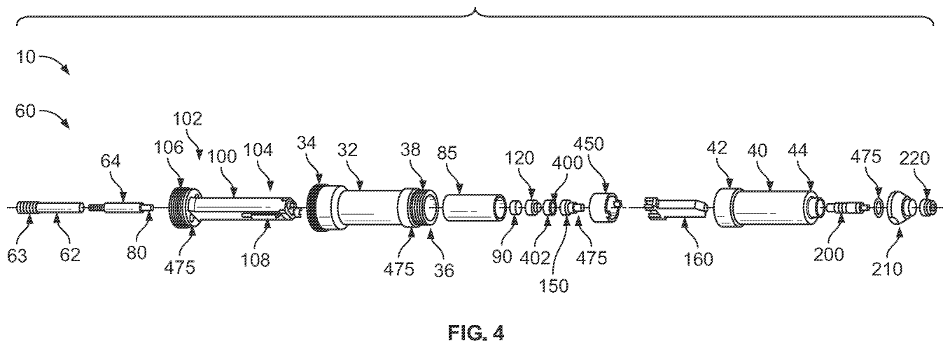

An example plasma arc discharge device for producing an NO-containing plasma stream is shown in FIGS. 1-4, although it will be appreciated that alternative sources of an NO-containing plasma stream may also be used to carrying out one or more of the disclosed methods.

Referring to FIGS. 1-4, the improved plasma arc discharge device 10 for producing and delivering NO-containing plasma stream is shown. The plasma arc discharge device 10 may form an NO-containing plasma stream for treating a biologic object. Additional details of the plasma arc discharge device 10 are included in U.S. Pat. No. 10,850,250 to Paris et al., assigned to Origin, Inc., and U.S. Patent Application Publication No. 2021/0077975 to Paris et al., assigned to Origin, Inc., the entirety of which are incorporated herein by reference. The plasma arc discharge device 10 may form the NO-containing plasma stream by using a DC arc discharge to generate NO in an interelectrode area 500 (e.g., an area between a cathode and an anode). As will be described in greater detail, the device 10 may be configured to produce and release, from a tip of the plasma arc discharge device 10, various concentrations of nitric oxide (“NO”) in more consistent output as compared to prior devices. Specifically, the plasma arc discharge device 10 may, because of one or more specifically engineered confluences of high voltage electrical arc-discharge, high velocity air impingement, and recirculating cooling fluid, produce various concentrations of NO-containing plasma stream as compared to prior art devices.

The device 10 includes an anode 20, a cathode 60, an interim electrode 120 for producing NO-containing plasma stream, and an NO-containing plasma stream flow outlet channel 152 leading from the interelectrode area 500 to a nozzle 200 for directing and releasing the NO-containing plasma stream flow from the plasma arc discharge device 10. As illustrated, the anode 20 may include a proximal anode 150, a coolant divider 160, and the nozzle 200.

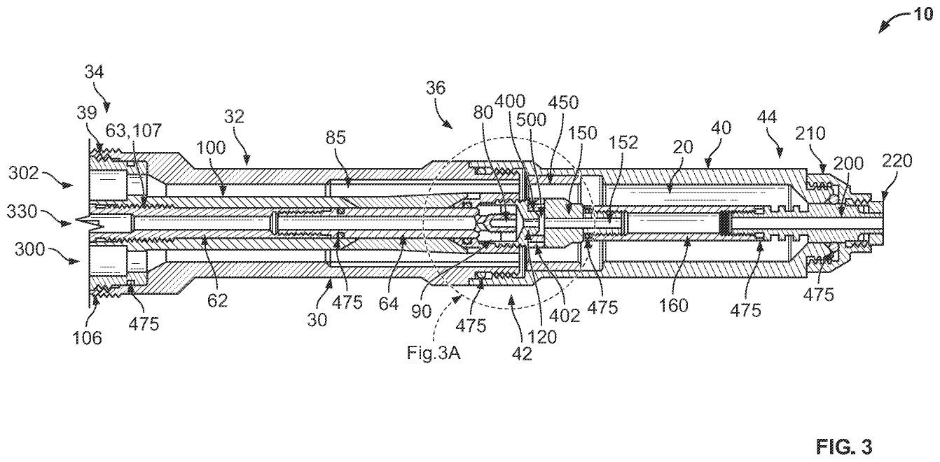

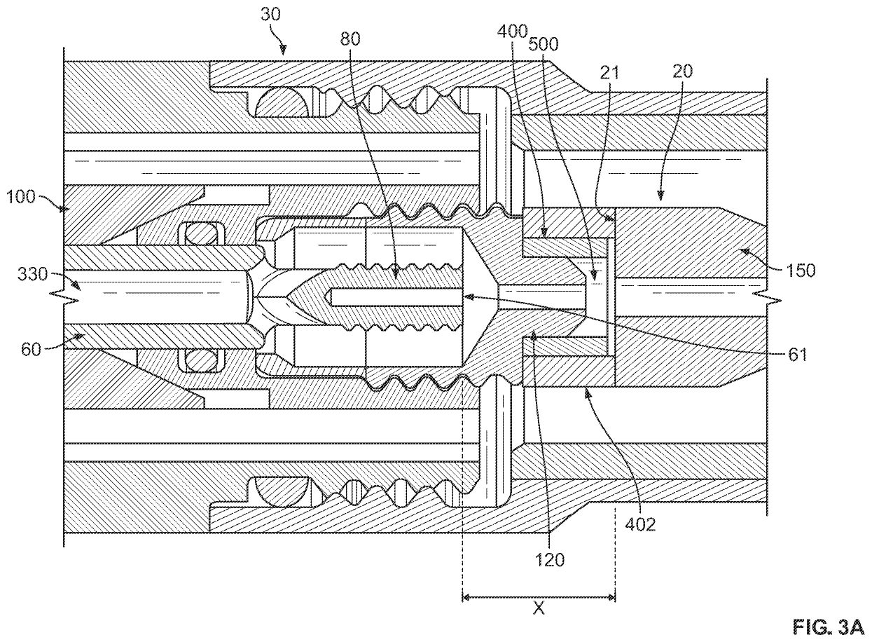

The position of the anode 20 with respect to the cathode 60 can be adjusted to produce various concentrations of NO-containing gas. That is, referring to FIG. 3A, according to one aspect of the present disclosure, the anode 20, and more specifically, the proximal anode 150, may have a proximal surface 21. In addition, the cathode 60, and more specifically, the hafnium tip 80 may have a distal surface 61. The longitudinal distance X, as measured between the distal end 61 of the cathode 60 and the proximal surface 21 of the anode 20, can be varied to produce different NO concentrations. As will be understood, varying the offset or distance X between the distal end 61 of the cathode 60 and the proximal surface 21 of the anode 20, varies the amount of voltage needed to be applied to the cathode 60 to maintain the plasma arc, and thus varies the NO concentration in the gas. In use, the distance X between the distal end 61 of the cathode 60 and the proximal surface 21 of the anode 20 can be adjusted by any suitable mechanism now known or hereafter developed.

The plasma arc discharge device 10 may also include an outer housing 30. The outer housing 30 may comprise the exterior housing or enclosure of the plasma arc discharge device 10. In addition, in use and as will be described in greater detail below, the outer housing 30 acts or receives an electrical ground. Moreover, the outer housing 30 acts as a container or housing for containing and directing fluid within the plasma arc discharge device 10. In the illustrated embodiment, the outer housing 30 may be formed from two components, e.g., a lower shell 32 and an upper shell 40, for ease of assembly. As will be described in greater detail below, the lower shell 32 and the upper shell 40 may be threadably coupled to one another, although it is envisioned that they may be coupled by other means including being manufactured as a single component.

The cathode 60 may be formed as a single component. Alternatively, the cathode 60 may be manufactured from multiple components, which are then coupled together. As illustrated, the cathode 60 may include a proximal cathode 62 and a distal cathode 64. In use, the cathode 60 may be centrally located within the lower shell 32. As illustrated, the proximal cathode 62 and the distal cathode 64 may be threadably coupled to one another, although it is envisioned that they may be coupled by any other means including, for example, welding, press-fit, etc.

In one embodiment, the cathode 60 may also include a hafnium tip 80 associated with the distal cathode 64. The hafnium tip 80 may be coupled to the cathode 60 (e.g., distal cathode 64) by any means including, for example, a press fit. In use, the hafnium tip 80 acts to provide or release electrons to stabilize or maintain the arc. Alternatively, it is envisioned that metals other than hafnium may also be used. Moreover, in an alternate embodiment, it is envisioned that the cathode may be “tip-less”, for example, for high-voltage/low-current versions of the device. As previously mentioned, the position of the cathode 60 with respect to the anode 20 may be adjusted to produce various concentrations of NO-containing gas. That is, as will be described in greater detail, in one embodiment, the distance between the cathode 60 and the anode 20 may be adjusted to produce different concentrations of NO-containing gas.

The plasma arc discharge device 10 may also include an insulator 100 positioned between at least a portion of the cathode 60 and at least a portion of the outer housing 30, for example, the lower shell 32, to insulate at least a portion of the outer housing 30 from the cathode 60. As will be described in greater detail below, an interim electrode 120 may be positioned at a distal end of the insulator 100. As will be described in greater detail below, the insulator 100 may be coupled via, for example, a threaded coupling to the outer housing 30 (e.g., at a proximal end 34 of the lower shell 32). In addition, the insulator 100 may be coupled via, for example, a threaded coupling to the cathode 60 (e.g., at proximal end of the proximal cathode 62). By providing a threaded connection between the cathode 60 (e.g., the proximal cathode 62) and the insulator 100, adjustment of the relative position of the cathode 60 (and hence the hafnium tip 80 coupled to a distal end thereof) with respect to the interim electrode 120, which is coupled to the distal end of the insulator 100, can be adjustably varied to, for example, vary the NO concentration. As such, in use, the insulator 100 helps fix the spacing between the interim electrode 120 and the cathode 60. In use, when assembled, the interim electrode 120 may be positioned within or adjacent to the anode 20 (e.g., the proximal anode 150) and the cathode 60 to provide ignition and stability of the electrical arc between the anode 20 and the cathode 60.

In use, a high voltage supply can be connected to the cathode 60 while the outer housing 30 may be connected to ground. In addition, the outer housing 30 may be in electrical contact with the anode 20, for example, the upper shell 40 may be in electrical contact with the nozzle tip 200, thus enabling the anode 20 to be electrically grounded so that, in use, an arc is generated in the interelectrode area 500 (e.g., area between the cathode 60 and anode 20). That is, in use, the nozzle tip 200, the nozzle tip driver 220, and a jacket assembly 210 associated with the nozzle tip 200 may be in contact with the outer housing 30 and thus electrically grounded. The grounding may be electrically transferred to the coolant divider 160 and the proximal anode 150, which may also be grounded as a result, so that the arc is generated in the interelectrode area 500 between the cathode 60 and the anode 20. As previously mentioned, the insulator 100 may be positioned between the cathode 60 and a portion of the outer housing 30 (e.g., between the cathode 60 and a portion of the lower shell 32) to insulate at least a portion of the outer housing 30 from the cathode 60. In addition, the anode 20 and the cathode 60 are electrically insulated with respect to the interim electrode 120, which is located adjacent the hafnium tip 80. As such, the interim electrode 120 is said to have a “floating” potential. By this arrangement, the electrical arc occurs at the juncture between the interim electrode 120 and the hafnium tip 80, emanating into the interelectrode area 500. Upon being generated in the interelectrode area 500, the NO-containing gas is directed thru the NO-containing gas flow outlet channel 152 to the nozzle tip 200. Specifically, the NO-containing gas passes thru respective output flow channels formed in the proximal anode 150, the coolant divider 160 and the nozzle tip 200, which output flow channels are in line with the cathode 60 to provide the output flow of the NO-containing gas through the nozzle tip 200.

The plasma arc discharge device 10 may use any method now known or hereafter developed in order to create the DC arc discharge in the interelectrode area 500 (e.g., area between the cathode 60 and the anode 20). For example, the plasma arc discharge device 10 may include a voltage supply for applying a voltage to the cathode 60 to generate and maintain a DC arc discharge between them, wherein a positive potential is applied to the outer housing 30 and a negative potential is applied to the cathode 60. The proximal anode 150 may be in fluid communication with a pathway 330 (e.g., a central hollow portion) formed in the cathode 60 for injecting a source gas into the interelectrode area 500 (e.g., area between the cathode 60 and the anode 20), where the source gas contains at least oxygen and nitrogen. The interelectrode area 500 may also be in fluid communication with the NO-containing gas flow outlet channel 152 formed in the anode 20 (e.g., the proximal anode 150, the coolant divider 160 and the nozzle tip 200) for directing the NO-containing gas flow from the interelectrode area 500. The NO-containing gas flow outlet channel 152 may further direct the NO-containing gas flow to the nozzle tip 200 so that the NO-containing gas flow can be used to treat a biologic object. As will be understood, the NO-containing gas flow is formed in the interelectrode area 500 between the anode 20 (e.g., proximal anode 150), the interim electrode 120, and the cathode 60 from a source gas under the effect of a DC arc discharge generated and maintained in the interelectrode area 500 between the anode 20 (e.g., proximal anode 150) and the cathode 60.

The arc discharge between the anode 20 and the cathode 60 may be generated by providing an open-circuit DC voltage across the cathode 60 and forming one or a series of high-voltage pulses to generate a spark discharge between the anode 20 and cathode 60. A value of the open-circuit voltage may be selected and adjusted to provide the change of the spark discharge to a stationary arc discharge. In non-limiting exemplary embodiments, the open-circuit voltage can be at least 200 V, and the high-voltage pulse can be at least 4 kV although other value combinations of open and high pulse voltages may be implemented. Furthermore, the stationary DC arc discharge may be maintained by a current of at least 1.8 A, where the arc discharge is stabilized using the interim electrode 120 to provide steady generation of plasma in the interelectrode area 500 (e.g., area between the cathode 60 and the anode 20) across the electric arc formed between the cathode 60 and the anode 20.

Referring again to FIG. 1, the tip of the plasma arc discharge device 10 may include a jacket assembly 210, a nozzle tip driver 220 and the nozzle tip 200 for releasing the NO-containing gas. In the illustrated embodiment, as will be described in greater detail below, the jacket assembly 210 may be in the form of a compression nut. In use, rotation of the nozzle tip driver 220 may advance or move the anode 20 (e.g., the nozzle tip 200, the coolant divider 160, and the proximal anode 150) towards the interim electrode 120 and the cathode 60 until the anode 20 (e.g., proximal anode 150) contacts a chamber standoff, spacer or cylinder (used interchangeably herein without the intent to limit) 400 disposed between the interim electrode 120 and the anode 20 (e.g., proximal anode 150) to ensure a precise distance between the interim electrode 120 and the anode 20 (e.g., proximal anode 150).

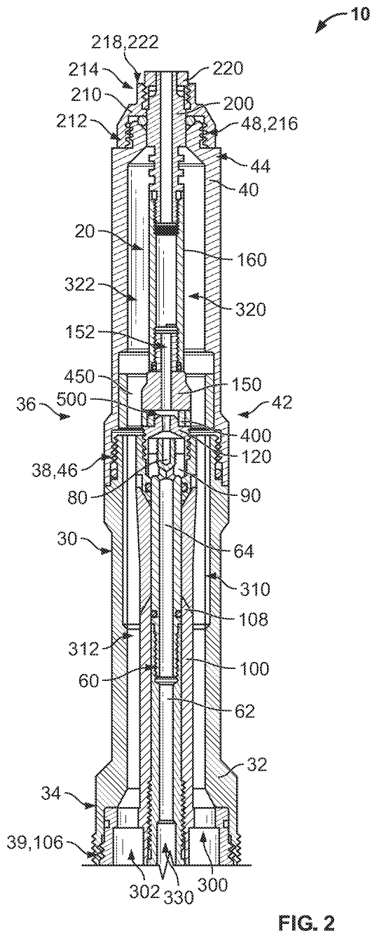

As shown in FIGS. 2-4, and as previously mentioned, the outer housing 30 may be formed from two components, a lower shell 32 and an upper shell 40. The lower shell 32 may include a proximal end 34 and a distal end 36. Similarly, the upper shell 40 may include a proximal end 42 and a distal end 44. The distal end 36 of the outer housing 30 may include a plurality of threads 38 for engaging a corresponding plurality of threads 46 formed on the proximal end 42 of the upper shell 40. As previously mentioned, the proximal end 34 of the outer housing 30 may also include a plurality of internal threads 39 for engaging external threads 106 formed on a proximal end 102 of the insulator 100. In addition, the insulator 100 may include a plurality of internal threads 107 for engaging external threads 63 formed on the cathode 60 (e.g., proximal cathode 62). In use, by threadably coupling the cathode 60 to the insulator 100, a user is able to adjust the relative position of the cathode 60 (and hence the hafnium tip 80 coupled to a distal end thereof) with respect to the interim electrode 120, which is coupled to the distal end of the insulator 100, and with respect to the anode 20, to achieve a desired NO concentration. For example, in use, a user can engage a proximal end of the cathode 60 via, for example, a tool such as, but not limited to, a screwdriver, a socket drive, etc. and by rotating the cathode 60 adjust the position of the cathode 60 with respect to the anode 20 and the interim electrode 120.

In addition, the jacket assembly 210 may include a proximal end 212 and a distal end 214. The distal end 44 of the upper shell 40 may include a plurality of threads 48 for engaging a corresponding plurality of threads 216 formed on the proximal end 212 of the jacket assembly 210. Similarly, the nozzle tip driver 220 may include a plurality of threads 222 for engaging a corresponding plurality of threads 218 formed on the distal end 214 of the jacket assembly 210. Tightening or loosening of the nozzle tip driver 220 when threaded in the jacket assembly 210 can allow for adjustable positioning of the anode 20 relative to the cathode 60 in order to produce various desired NO concentrations. It should be understood that while the lower shell 32, the upper shell 40, the external threads 106 formed on the insulator 100, the jacket assembly 210 and the nozzle tip driver 220 have been described and illustrated as including a plurality of threads for engaging one another, it is contemplated that the components may alternatively be coupled together by any other means now known or hereafter developed including, for example, being constructed as a single unitary component.

As previously mentioned, the lower shell 32 may enclose the cathode 60 (e.g., proximal cathode 62, distal cathode 64, and hafnium tip 80). In addition, the outer housing 30, and more specifically, the lower shell 32, may further enclose the interim electrode 120 and the insulator 100. The insulator 100 may be made from a dielectric material, such as, for example, a polymer, a ceramic, PTFE (Polytetrafluoroethylene), etc. The insulator 100 may be positioned between the cathode 60 and the outer housing 30, for example, between the cathode 60 and the lower shell 32 to insulate the cathode 60 and the lower shell 32 from one another. In addition, as previously mentioned, the insulator 100 also isolates the cathode 60 (e.g., the proximal and distal cathodes 62, 64) from the interim electrode 120. The interim electrode 120 is used to initiate the arc and maintain/stabilize the continuity of electrical discharge from the cathode 60 to the anode 20.

The plasma arc discharge device 10, and more specifically the lower shell 32, may also include a cathode insulator 85 for providing a dielectric barrier between the outer housing 30 (e.g., lower shell 32) and the cathode 60 for providing sufficient electrical insulation.

The outer housing 30, and more specifically, the upper shell 40, may enclose the anode 20 such as the proximal anode 150, the coolant divider 160 and the nozzle tip 200. In use, when the high voltage supply is connected to the cathode 60, an electrical arc is produced between the interim electrode 120 and the cathode 60 (e.g., hafnium tip 80) and onto the anode 20 thus creating the NO-containing gas plasma in the interelectrode area 500 (e.g., area between the cathode 60 and the anode 20) across the electric arc formed between the cathode 60 and the anode 20. Thereafter, the NO-containing gas is directed through the output flow channels formed in the proximal anode 150, the coolant divider 160, and the nozzle tip 200, all of which are in line with the cathode 60. In this way, an output flow of the NO-containing gas is discharged through the nozzle tip 200.

As previously mentioned, the plasma arc discharge device 10 may include one or more pathways for receiving fluid therein. Referring to FIGS. 2 and 3, in one embodiment, the plasma arc discharge device 10 and more specifically the lower shell 32 and the insulator 100 may form first and second lower coolant pathways 310, 312 coupled, respectively, to a fluid entry port 300 and a fluid exit port 302. Similarly, the device 10, and more specifically the upper shell 40 and the coolant divider 160 may form first and second upper coolant pathways 320, 322 which are in fluid communication with the first and second lower coolant pathways 310, 312, respectively. In use, when the lower shell 32 is coupled to the upper shell 40, the first and second lower coolant pathways 310, 312 are aligned with the first and second upper coolant pathways 320, 322. In this manner, a fluid (e.g., liquid, gas) may be introduced into the plasma arc discharge device 10 via the fluid entry port 300, circulated thru the plasma arc discharge device 10 via the first lower coolant pathway 310 and the first upper coolant pathway 320 located between the lower and upper shells 32, 40 and the cathode 60 and anode 20, respectively. At the distal end of the upper shell 40, the cooling fluid may be passed to the second upper coolant pathway 322, then to the second lower coolant pathway 312, where it exits through the fluid exit port 302 located at the proximal end 34 of the insulator 100. More specifically, the cooling fluid may be introduced into the device 10 at the fluid entry port 300 located at the proximal end 34 of the lower shell 32. The cooling fluid may travel the fluid pathways formed in the lower shell 32 and into the upper shell 40 adjacent to the coolant divider 160, then back down and out of the device 10 via the fluid exit port 302 located at the proximal end 34 of the lower shell 32.

In general, as the cooling fluid travels through the coolant pathways formed within the outer housing 30, heat from the NO-containing gas is transferred through the coolant divider 160 and into the cooling fluid, reducing the temperature of the NO-containing gas as it travels to the nozzle tip 200. As will be appreciated, the cooling fluid also removes heat from the anode itself.

The insulator 100 may also include one or more coolant entryways 108 for providing a pathway for the cooling fluid to interact with the cathode 60. That is, the insulator 100 may include one or more coolant entryways 108 for providing a pathway for the cooling fluid to directly contact at least a portion of the cathode 60 (e.g., distal cathode 64) prior to the interim electrode 120. The configuration of these coolant entryways 108 is designed to maximize thermal draw from the cathode 60 to the cooling fluid thereby reducing the operating temperature of the cathode 60 while under load.

In one embodiment, the fluid could be electrically conductive. In contrast, dielectric fluid, such as ethylene glycol, propylene glycol, or silicone oil may be used to maximize thermal draw and to prevent the interim electrode 120 from achieving electrical potential from the cathode 60.

As illustrated, the insulator 100, the coolant divider 160, and other components located adjacent the coolant pathways 310, 320, 322, 312 may have a substantially flat or ribbed shape or surface area to allow for increased cooling of the internal components. In this manner, the contact area is maximized between the insulator 100, the coolant divider 160, and the cooling fluid, thereby enhancing heat transfer (e.g., cooling efficiency) therebetween.

In contrast with prior devices that provide cooling beyond the nozzle tip (i.e., most of the cooling of the NO-containing gas occurs after the NO-containing gas has exited the nozzle tip), the disclosed plasma arc discharge device 10 enables cooling, via the cooling fluid, to begin within the plasma arc discharge device 10 such as, for example, in the proximal anode 150. That is, according to one aspect of the present disclosure, the cooling fluid may interact with and cool the NO-containing gas along a substantial length of the NO-containing gas flow outlet channel 152 between the proximal anode 150 to the nozzle tip 200. In this manner, the exiting NO-containing gas is cooled to a much greater extent before it exits the plasma arc discharge device 10 as compared to prior devices. Due to the reduced temperature of the NO-containing gas exiting the nozzle tip 200, the concentration of NO-containing gas that can be immediately directed to the treatment site, which can be higher as compared to prior devices. In non-limiting exemplary embodiments, the NO-containing gas immediately exiting the nozzle tip 200 may approach 1000° C. and exceed 20,000 ppm of NO, which may result in the NO-containing gas at the treatment site being approximately 50° C. or less and 700 ppm to 1,1000 ppm of NO.

As previously mentioned, the cathode 60 may further include a central hollow portion that provides a pathway 330 for air to be forced through the cathode 60 and into the interelectrode area 500 for use in generating a plasma in the interelectrode area 500. Referring to FIGS. 1-4, in one embodiment, the distal end of the cathode 60 may include one or more tangential holes. In use, the holes transfer the air around the hafnium tip 80 and into the interelectrode area 500. The tangential holes may be configured to create a vortex in the airflow as the gas passes through the interelectrode area 500. That is, in connection with the example embodiment of the cathode tip illustrated in FIGS. 1-4, as air comes in through the central hollow portion and out the holes, the air swirls around the cathode 60 (e.g., hafnium tip 80). The swirling airflow surrounds the plasma and its afterglow as it goes out through the interim electrode 120 and the anode 20, which assists in in arc stabilization, resulting in a more consistent NO production.

In use, an air pump (not shown) may supply forced air through the proximal end 34 of the plasma arc discharge device 10 (via pathway 330) into the cathode 60 and forced out the nozzle tip 200. This allows the NO-containing gas created in the interelectrode area 500 to be forced up and through the nozzle tip 200 to deliver it to the treatment area.

Classical thermodynamics confirmed by compositional analysis of the thermodynamical equilibrium of air passing through plasma generator shows that at a temperature lower than 2000° C. the concentration of NO in the gas does not exceed 1%. Increasing the temperature of the plasma discharge increases the NO concentration up to its maximum (˜5%) at a temperature of 3500-4000° C. Slightly less than 4000° C. is the temperature of the electrical discharge in the plasma arc of the illustrated plasma arc discharge device 10. Plasma-chemical reactions, which lead to the formation of NO, can be expressed by the following chemical formula:

N₂+O₂→2NO-180.9 kJ

The lifetime of the NO molecule at high temperatures is comparable to the time of its synthesis because of the fast reaction of recombination:

2NO+O₂→2NO₂

As will be appreciated, the NO-containing gas stream together with other synergistic components generated by a plasma device can be used for a variety of purposes. For example, the stream can serve as an antimicrobial agent. In addition, NO-containing gas stream can be used to facilitate hair-growth, as an anti-wrinkle agent, to reduce inflammation, or to facilitate vasodilation. The NO-containing gas stream further can be employed to alleviate pain associated with osteoarthritis and rheumatoid arthritis, i.e., OA and RA. It can also be effective in combating gram-positive microorganisms, gram-negative microorganisms, fungi (including onychomycosis), and viruses. It is also therapeutic in treating osteoporosis, collagen formation, stem cell signaling, satellite cell differentiation, wound-healing, wound-management, reduction in scar tissue, remediation of activity-related injury, and acne. A plasma generated NO-containing gas stream together with other components present in the stream can also aid in nerve regeneration, inhibit cancer cell proliferation, promote apoptosis, and stimulate endogenous nitric oxide production.

In practice, the NO-containing stream of gas together with other components present in the stream can be applied directly to or adjacent to living tissue in order to produce the desired effect. It can effectively function to maintain homeostasis in the cardiovascular and respiratory systems. NO, as a signaling molecule, can cause vasodilation which promotes blood vessel flexibility, eases blood pressure, cleans the blood, reverses atherosclerosis, effectively prevents cardiovascular diseases and aids in recovery therefrom. Another important function of NO is slowing down atherosclerotic plaque deposition on vascular walls. NO also plays an active defense role in the immune system. It is a strong antioxidant, and can suppress bacterial infections, viruses, and parasitic attacks. It can even deter some types of cancer cell growth. In patients with moderate to severe diabetes, NO can prevent many common and serious complications. NO can also significantly reduce the pain associated with joint swelling in arthritis. NO can effectively decrease the risk of cancer, diabetes, myocardial infarction, and stroke.

In the nervous and endocrine systems, NO can induce normal functioning of various body organs. NO can permeate freely through the cell membrane for biological signaling, adjust cellular activities and lead every organ to complete its function properly, including the lungs, liver, kidneys, stomach, heart, brain, and genitals. NO can increase blood flow to the genital organs to maintain normal sexual function. The brain transmits signals via its surrounding nerves to the perineal region to provide it with sufficient NO to cause vascular dilation, increasing blood flow to enhance erectile function. Under some conditions, weak erections are the results of insufficient NO production by nerve endings.

NO can also slow the aging process and improve memory. The NO molecules produced by the immune system are not only capable of destroying invading microorganisms, but also help activate and nourish brain cells, significantly slowing aging and improving memory. See, for example, S Moncada, Nitric oxide: discovery and impact on clinical medicine. J R Soc Med. 1999 April; 92(4): 164-169.

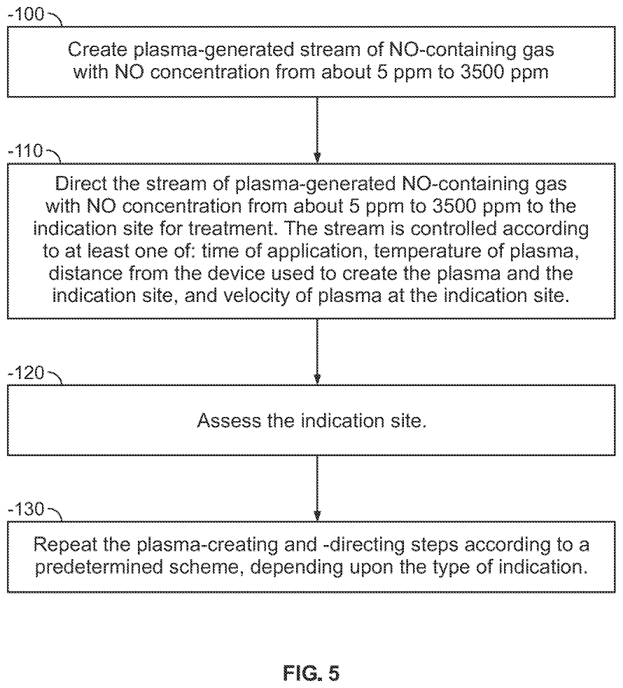

Referring now to FIG. 5, a flow diagram illustrating an exemplary method for administering NO in a plasma state to a treatment site in accordance with the present disclosure is shown. At a first step 100 of the exemplary method, a discrete stream of matter that has been put into a state of plasma may be created, in which the stream has, as part of its content, NO in a concentration from about 5 ppm to 3,500 ppm. At step 110, the NO-containing plasma stream is directed at an indication site in living organism, where the stream is controlled according to at least one of time of application, temperature of the matter in a plasma state, distance from device used to create the matter in a plasma state and the indication site, and velocity of matter in a plasma state at the indication site. At step 120, the indication site is assessed. At step 130, the creating and directing steps are repeated according to a predetermined scheme, depending upon the type of indication.

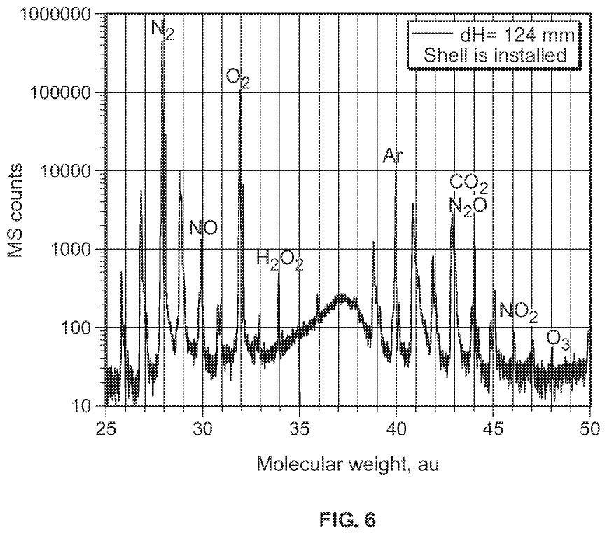

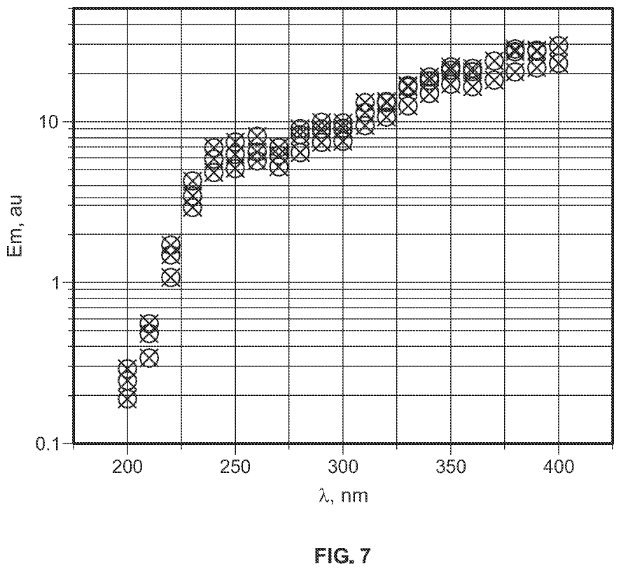

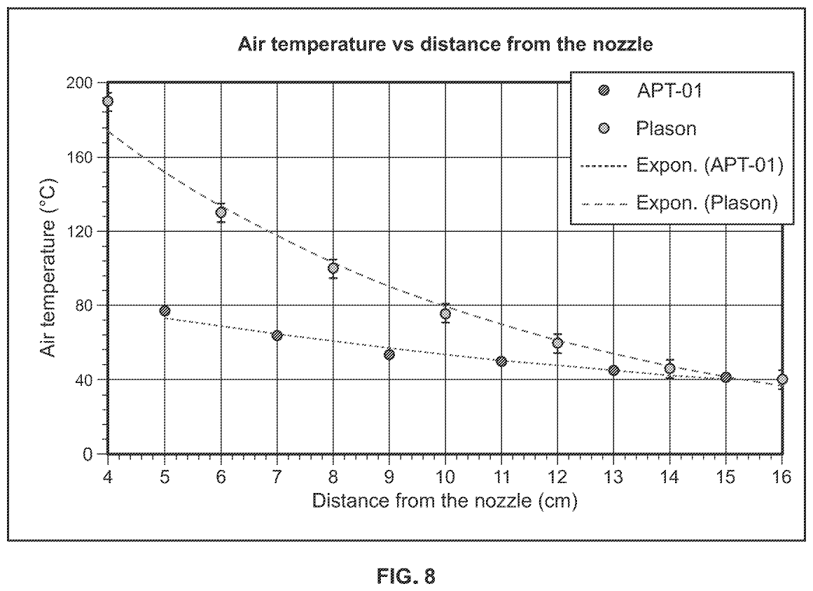

FIG. 6 illustrates mass-spectroscopy spectra showing concentrations of different components measured at different distances from the tip of nozzle 200 in an exemplary plasma arc discharge device 10. The spike visible at 34 au of H₂O₂ is higher at larger distances from the discharge nozzle of the device, at 84 mm and 124 mm due to the recombination of the radicals OH, OH+OH=H₂O₂. The hydroxyl radical, OH is a short-lived strong bactericidal specie, which also contributes to the increase of a number of bactericidal H₂O₂ molecules.

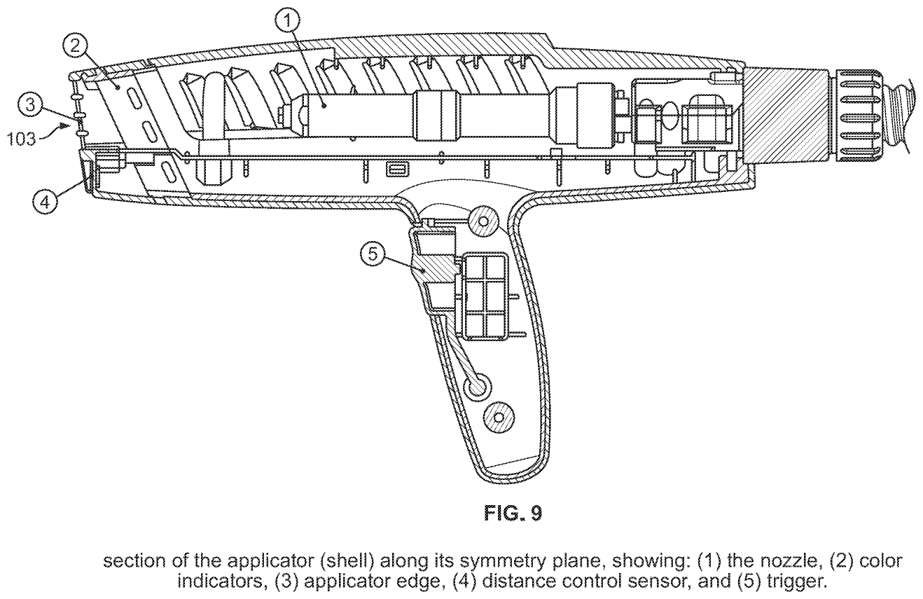

Table 1 provides measured concentrations of the stable components in the plasma stream emitted by the exemplary plasma arc discharge device. Concentration changes from almost 10,000 ppm near the plasma arc nozzle exit to 850-900 ppm at a distance of 40 mm from the edge of the safety shell 103 of the applicator (see FIG. 9) of the exemplary plasma arc discharge device.

TABLE 1
Concentrations of different components of NO-rich
stream measured using mass-spectral analysis.

| | Distance from the tip | | |
|---|---|---|---|
| Stream | of the nozzle (mm) | | |
| Composition | 10 | 84 | 124 |
| N₂ | 7.66E−01 | 7.65E−01 | 7.63E−01 |
| O₂ | 1.90E−01 | 2.00E−01 | 2.01E−01 |
| Ar | 3.30E−02 | 3.40E−02 | 3.42E−02 |
| NO | 9.97E−03 | 9.01E−04 | 8.49E−04 |
| NO₂ | 1.13E−04 | 6.80E−05 | 7.00E−05 |
| H₂O₂ | 4.30E−05 | 8.20E−05 | 9.70E−05 |

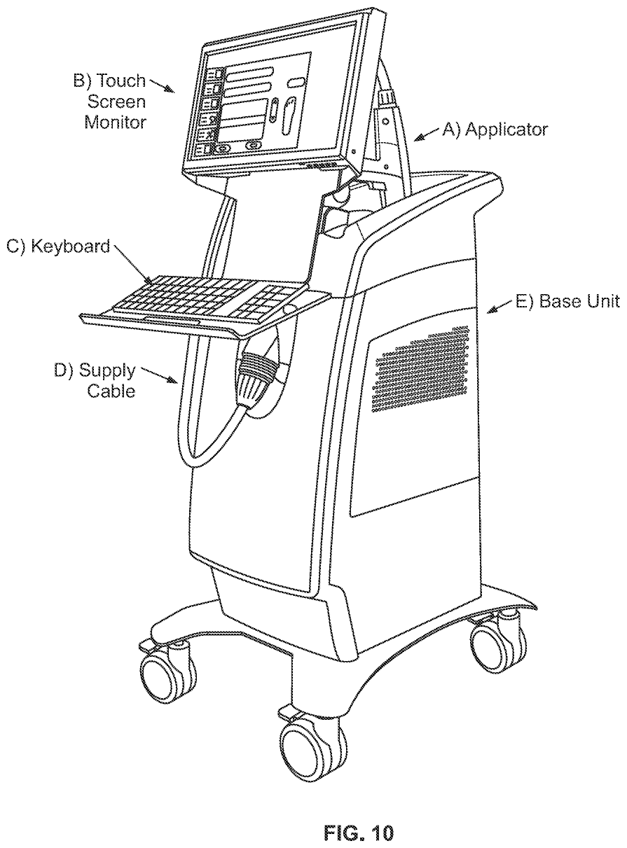

FIG. 10 is a graph illustrating a spectral distribution of UV radiation from a plasma arc emitted from the plasma arc torch of FIGS. 1-4. The figure shows a relative spectral photon flux distribution in the range 200-400 nm. Spectral step was 10 nm, and the signal is averaged over 5 ms (500,000 sampling points per each mark). UV-radiation in the shown part of the spectrum is known to have significant bactericidal effect thus contributing to the healing effect in using the invented device/devices.

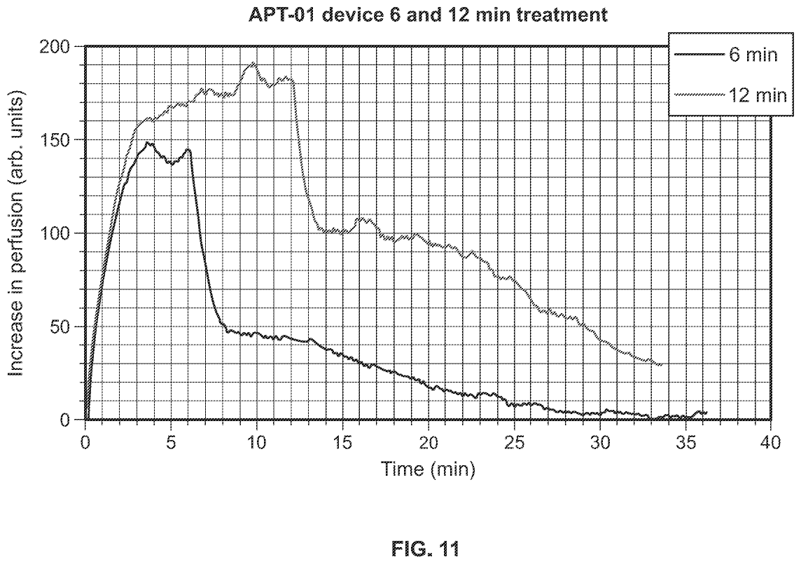

FIG. 11 is a graph illustrating the temperature of the NO-containing plasma stream emitted by the device 10 of FIGS. 1-4, 9, and 10. Mass-spectroscopy and UV emission analyses were performed for one embodiment of the exemplary plasma-generating device shown in FIGS. 9 and 10. As previously described, the device 10 developed by Origin Inc. (see, e.g., FIGS. 1-4, 9 and 10), is a next generation plasma device that operates using plasma arc discharge. This new device 10 is being used for a number of medical indications listed below, in particular for infected wound healing, in treating chronic diabetic foot ulcers (DFU). The device consists of base unit (FIG. 10), touch screen monitor, keyboard, supply cable and applicator (FIG. 9). The applicator contains the device 10 (FIGS. 1-4) and provides a set distance control sensor to maintain acceptable temperatures and therapeutic concentration of NO-rich gas stream at the target treatment area. It also includes color indicators, which communicate to the user if the device is located at the proper distance from biological surface. The design of the new device provides for control of physical and chemical parameters for a particular treatment protocol, which is configurable by the user.

NO, as delivered by the device 10, has been found by the inventors to stimulate a prolonged activity as determined by recording subdermal blood flow measured by speckled Doppler measurements, which appears to be dose dependent. FIG. 11 illustrates a recording of forearm blood profusion during and after 6- and 12-minute exposures to plasma generated stream of NO using the device 10. Recordings of the heat effect on forearm blood flow have been well described. The vasodilation, predominently useful as a mechanism of thermoregulation and mostly due to NO release, is basically a square wave with the blood flow coming back to normal when the skin cools back to room air temperature.

Physical stimulation of the skin by sound or shock wave, NIR and lasers may all increase blood flow by causing endogenous NO release. For example, shock wave therapy has bee employed to treat chronic musculoskelital pain and others, such as Sanuwave, have studied shock wave for increasing healing. The inventors are unaware of any evaluation of enhanced blood flow prolongation, as shown in relation to FIG. 11, for any of these other technologies/techniques.

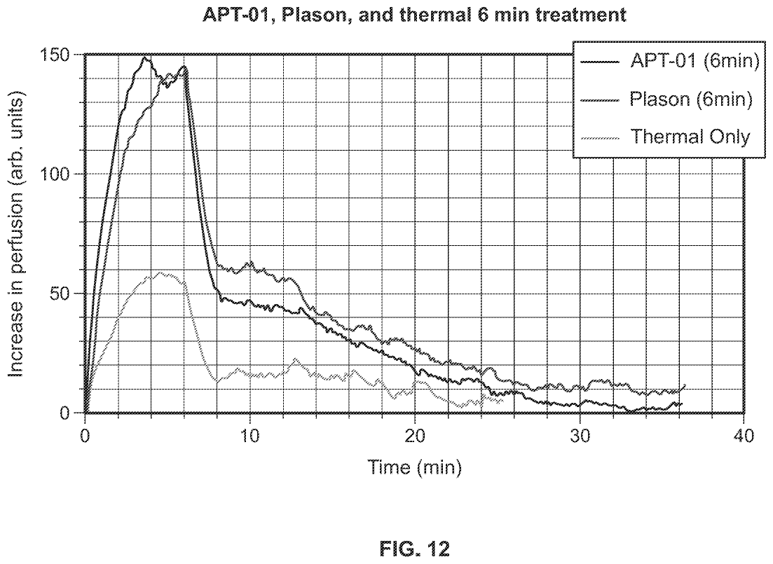

FIG. 12 illustrates a recording of forearm blood flow during and after 6-minute exposure to plasma generated stream of NO using the device 10. Three curves represent blood flow increase using heat gun, Plason, and the new APT-01 unit.

### Exemplary Indications

A non-limiting listing of exemplary indications for which the disclosed NO-containing stream of gas together with other stream components generated by a plasma source may find beneficial use as a treatment includes:
Infections
   Acute and Chronic Bacterial (gram-positive and -negative) skin and soft tissue infections
   Insect bites
   Abscesses
   Cellulitis
Fungal infections
   Tinea pedis
   Onychomycosis
   Candidiasis
   Other fungal skin infections
Viral infections
   Papilloma
   Herpetic infections (including Shingles)
   Molluscum contagiosum.
   Hand, foot and mouth disease
   Respiratory infections
Protozoal skin and soft tissue infestations
Wound-healing
   Chronic
      Diabetic foot ulcers
      Decubitus ulcers
      Venous stasis ulcers
      Sickle cell ulcers
      Vasculitic ulcers
      Haematologic ulcers
      Arterial ulcers
   Acute
   Sub-acute
   Radiation wounds
   Post-operative wounds
   Wounds with implanted medical devices and hardware
   Traumatic wounds of any etiology
   Chronic wounds of any etiology
   Insect bites
   Animal bites
   Burns (1^{st}-3^{rd} degree) partial or full thickness
Pain and inflammation
   Osteo-arthritis
      Knee
      Hand
      Ankle
      Shoulder
      Elbow
      Toe
      Spine and neck
      Hip
      Finger
   Rheumatoid arthritis
      Knee
      Hand
      Ankle
      Shoulder
      Elbow
      Toe
      Spine and neck
      Hip
      Finger
   Fasciitis
   Tendinitis
   Muscle spasms
Dermatology/Infectious-inflammatory
   Acne
   Roseola
   Non-specific exanthems
   Scar minimalization
   Hypertrophic scars
Ophthalmic
Hair loss
Dental
   Gingivitis
   Periodontitis
   Other intraoral conditions
Plastic Surgery
   Scar minimization
   Keloids
   Wrinkles

An exemplary baseline composition of a gas stream generated by a plasma source is shown in Table 2 below. It will be appreciated that this composition itemization is merely exemplary, and that other compositions can also be used to beneficial effect.

TABLE 2
Baseline plasma-generated stream composition.

| | |
|---|---|
| Stream | Concentration (vol. fraction) |

| Composition | Minimum | Maximum |
|---|---|---|
| NO | 2.00E−04 | 1.00E−03 |
| NO₂ | 0.00E+00 | 2.50E−05 |
| N₂ | 7.50E−01 | 7.80E−01 |
| O₂ | 1.80E−01 | 2.10E−01 |
| O₃ | 0.00E+00 | 1.00E−07 |
| H₂O₂ | 0.00E+00 | 1.50E−03 |
| H₂O | 0.00E+00 | 2.00E−02 |
| Ar | 1.00E−02 | 9.10E−01 |
| He | 5.20E−06 | 9.10E−01 |
| CO | 0.00E+00 | 5.00E−05 |
| CO₂ | 3.00E−04 | 5.00E−04 |
| H₂ | 0.00E+00 | 1.00E−02 |

An exemplary baseline treatment scheme is shown in Table 3 below. It will be appreciated that this baseline scheme may be adjusted, as will be described in relation to a number of examples to follow, to provide a desired treatment plan for an affected area and in response to a particular indication.

As shown in Table 3, the treatment variables include “distance from exit to site,” “time of application,” “number of treatments,” “length of time between treatments,” “temperature of plasma stream at contact with site,” and “velocity of plasma stream at contact with treatment site.”

“Distance from exit to site” will be understood to be the standoff distance, in centimeters, from the outlet of the plasma device (e.g., device 1, 10, 20) to the treatment site. “Time of application” will be understood to be the amount of time, in seconds, that the NO-containing matter in a plasma state will be directed from the plasma device onto the treatment site, per square centimeter of site area. Thus, the time of application will depend upon the size of the area being treated. “Number of treatments” will be understood to be the discrete number of treatments to be applied at the site. “Length of time between treatments” will be understood to be the amount of time elapsed between applications of the NO-containing stream of gas together with other stream components generated by a plasma source at the treatment site. “Temperature of plasma stream at contact with treatment site” will be understood to be the temperature of the NO-containing matter in a plasma state, in degrees Celsius, at the treatment site. “Velocity of plasma stream at contact with treatment site” will be understood to be the speed of the NO-containing stream of gas generated by a plasma source, in meters per second, at the treatment site. Minimum and maximum values are provided for each, recognizing that individual treatment specifications for particular indications will vary within the indicated ranges.

TABLE 3
Baseline treatment scheme.

| Parameter | Minimum | Maximum |
|---|---|---|
| Distance from exit to site (cm) | 1 | 25 |
| Time of application (s/cm²) | 5 | 45 |
| Number of treatments | 1 | 24 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

A series of exemplary specific treatment schemes will now be discussed in relation to various indications. These treatment schemes assume the use of matter in a plasma state having the compositions identified in Table 2.

### Example 1: Gram-Positive Bacteria Treatment Scheme

See Table 5 below, for partial list of gram-positive pathogens. See Table 6, below, for partial list of conditions that present with pathogens from Table 5.

The minimum treatment values and maximum treatment values are identified below are based on the severity of the gram-positive bacterial infection. Severity of the infection is determined by the surface area, depth, colony count and symptoms. Application of therapy increases in intensity, duration and frequency as the severity increases. Minimum treatment parameters define the requirements for the initiation of the decolonization process.

TABLE 4
Gram-positive bacteria treatment scheme.

| Parameter | Minimum | Maximum |
|---|---|---|
| Distance from exit to site (cm) | 1 | 25 |
| Time of application (s/cm²) | 5 | 45 |
| Number of treatments | 1 | 24 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

TABLE 5
gram-positive pathogens susceptible
to plasma-generated NO therapy.

| | |
|---|---|
| MRSA | Streptococcus mutans |
| MDSA | Mycobacterium tuberculosis |
| Staphylococcus aureus | Bacillus subtilis |
| Streptococcus A | Streptococcus pneumoniae |
| Streptococcus B | Vancomycin resistant enterococcus faecium |
| C. Difficile | |

TABLE 6
conditions that present with gram-positive pathogens
susceptible to plasma-generated NO therapy.

Abscesses
Actinomytosis
Animal bite infections
Burns
Carbuncles
Catheterizations
Cellulitis
Complicated SSTI
Decubitus ulcers
Dermatitis
Diabetic foot ulcers
Erysipelas
Erysipeloid
Folliculitis
Furuncles
Herpes Simplex
Herpetic Neuralgia
Impetigo
Keratolysis
Leishmaniasis
Myositis
Pressure ulcers
Pyodermas
Sickle cell ulcerations
Skin Flaps
Skin Grafts
Skin Tuberculosis
Surgical site infections
Traumatic wounds
Ulcerated lesions
Vasculitis
Venous ulcers

### Example 2: Gram-Negative Bacteria Treatment Scheme

See Table 8 for partial list of gram-negative pathogens. See Table 9 for partial list of conditions that present with pathogens from Table 8.

The minimum treatment values and maximum treatment values are based on the severity of the gram-negative bacterial infection. Severity of the infection is determined by the surface area, depth, colony count, and symptoms. Application of therapy increases in intensity, duration and frequency as the severity increases. Minimum treatment parameters define the requirements for the initiation of the decolonization process. Gram negative bacteria are more difficult to kill than gram positive, so longer treatments are required to decolonize (see Table 7).

TABLE 7
gram-negative bacteria treatment scheme.

| Parameter | Minimum | Maximum |
|---|---|---|
| Distance from exit to site (cm) | 1 | 25 |
| Time of Application (s/cm²) | 10 | 90 |
| Number of treatments | 1 | 24 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

TABLE 8
Gram-negative pathogens susceptible
to plasma-generated NO therapy.

| | |
|---|---|
| Escherichia coli | Stenotrophomonas maltopilia |
| Salmonella | Pseudomonas aeruginosa |
| Klebsiella pneumoniae | Acinetobacter baumannii |
| Serratia marcescens | Proteus Vulgaris |
| Enterobacter aerogenes | Pantoeaagglomerans |

TABLE 9
conditions that present with gram-negative pathogens
susceptible to plasma-generated NO therapy.

| | | |
|---|---|---|
| Abscesses | Erysipelas | Pyodermas |
| Actinomytosis | Erysipeloid | Sickle cell ulcerations |
| Animal bite infections | Folliculitis | Skin Flaps |
| Burns | Furuncles | Skin Grafts |
| Carbuncles | Herpes Simplex | Skin Tuberculosis |
| Catheterizations | Herpetic Neuralgia | Surgical site infections |
| Cellulitis | Impetigo | Traumatic wounds |
| Complicated SSTI | Keratolysis | Ulcerated lesions |
| Decubitus ulcers | Leishmaniasis | Vasculitis |
| Dermatitis | Myositis | Venous ulcers |
| Diabetic foot ulcers | Pressure ulcers | |

### Example 3: Wounds, Pressure Ulcers, Treatment Scheme

Wound severity classification subject to Table 11. Clinical presentation subject to Table 12.

The minimum treatment values and maximum treatment values are based on the severity of the pressure ulcer wound. Severity of the infection is determined by the surface area, depth, and symptoms. Application of therapy increases in intensity, duration and frequency as the severity increases. Minimum treatment parameters define the requirements for the initiation of the wound care management process.

TABLE 10
parameters for the wounds and pressure ulcers treatment scheme.

| Parameter | Minimum | Maximum |
|---|---|---|
| Distance from exit to site (cm) | 1 | 25 |
| Time of application (s/cm²) | 1 | 45 |
| Number of treatments | 1 | 200 |
| Length of time between treatments (hours | 3 | 168 |
| Temperature of plasma stream at contact | 10 | 60 |
| with site (° C.) | | |
| Velocity of plasma stream at contact | 0.5 | 200 |
| with site (m/s) | | |

TABLE 11
deep tissue wounds (pressure ulcer) classification.

| Stage | Description |
|---|---|
| I | Intact skin with non-blanchable redness usually over a bone |
| II | Partial thickness loss of dermis with open ulcer |
| III | Full thickness tissue loss; sub-cutaneous fat may be exposed but not |
| | bone, tendon, or muscle |
| IV | Full thickness tissue loss with exposed bone, tendon, or muscle |
| Unstageable | Full thickness tissue loss in which the base of the ulcer is covered by |
| | slough, until it is removed to expose the base of the wound the true |
| | depth and therefore stage cannot be determined. |

TABLE 12
other wound classification.

| | | |
|---|---|---|
| Acute | Dermal | Surgical |
| Chronic | Sub-dermal | Traumatic |

### Example 4: Wounds, Neuropathic Ulcers Treatment Scheme

Severity classification subject to Table 14. Clinical presentation subject to Table 15.

The minimum treatment values and maximum treatment values are based on the severity of the neuropathic ulcer wound. Severity of the wound is determined by the surface area, depth, and symptoms. Application of therapy increases in intensity, duration and frequency as the severity increases. Minimum treatment parameters define the requirements for the initiation of the wound care management process.

TABLE 13
neuropathic ulcers treatment scheme.

| Parameter | Minimum | Maximum |
|---|---|---|
| Distance from exit to site (cm) | 1 | 25 |
| Time of application (s/cm²) | 5 | 90 |
| Number of treatments | 1 | 200 |
| Length of time between treatments (hours) | 3 | 168 |
| Temperature of plasma stream at contact | 10 | 60 |
| with site (° C.) | | |
| Velocity of plasma stream at contact | 0.5 | 200 |
| with site (m/s) | | |

TABLE 14
neuropathic ulcers severity classification.

| Grade | Description |
|---|---|
| I | Superficial ulcer |
| II | Penetration into tendon or joint capsule |
| III | Involvement of deeper tissues |
| IV | Gangrene of the forefoot |
| V | Gangrene involving more than two-thirds of the foot |

TABLE 15
Other wound classifications.

| | | |
|---|---|---|
| Acute | Dermal | Surgical |
| Chronic | Sub-dermal | Traumatic |

### Example 5: Wounds, Venous Ulcers Treatment Scheme

Severity classification is subject to Table 17. Clinical presentation is subject to Table 18.

The minimum treatment values and maximum treatment values are based on the severity of the pressure venous wound. Severity of the wound is determined by the surface area, depth, and symptoms. Application of therapy increases in intensity, duration and frequency as the severity increases. Minimum treatment parameters define the requirements for the initiation of the wound care management process. Treatment includes a border around the wound site of up to 4 cm due to circulatory issues.

TABLE 16
parameters for the venous ulcers treatment scheme.

| Parameter | Minimum | Maximum |
|---|---|---|
| Distance from exit to site (cm) | 1 | 25 |
| Time of application (s/cm²) | 5 | 45 |
| Number of treatments | 1 | 24 |
| Length of time between treatments (hours) | 3 | 168 |
| Temperature of plasma stream at contact | 10 | 60 |
| with site (° C.) | | |
| Velocity of plasma stream at contact | 0.5 | 200 |
| with site (m/s) | | |

TABLE 17
venous ulcers severity classification.

| Grade | Description |
|---|---|
| I | Superficial ulcer |
| II | Penetration into tendon or joint capsule |
| III | Involvement of deeper tissues |
| IV | Gangrene of the forefoot |
| V | Gangrene involving more than two-thirds of the foot |

TABLE 18
other wound classification.

| | | |
|---|---|---|
| Acute | Dermal | Surgical |
| Chronic | Sub-dermal | Traumatic |

### Example 6: Wounds, Burns Treatment Scheme

Severity classification is subject to Table 20.

The minimum treatment values and maximum treatment values are based on the severity of the burn. Severity of the burn is determined by the surface area, depth, and symptoms. Application of therapy increases in intensity, duration and frequency as the severity increases. Distance from the burn site dependent on patient's pain threshold. Minimum treatment parameters define the requirements for the initiation of the burn care management process.

TABLE 19
burns treatment scheme.

| Parameter | Minimum | Maximum |
|---|---|---|
| Distance from exit to site (cm) | 10 | 30 |
| Time of application (s/cm²) | 10 | 90 |
| Number of treatments | 1 | 200 |
| Length of time between treatments (hours) | 1 | 168 |
| Temperature of plasma stream at contact | 10 | 50 |
| with site (° C.) | | |
| Velocity of plasma stream at contact | 0.5 | 200 |
| with site (m/s) | | |

TABLE 20
burn wounds severity classification.

| Stage | Description |
|---|---|
| I | Superficial |
| | Superficial partial thickness skin loss |
| III | Deep partial thickness skin loss |
| IV | Full thickness dermal |
| V | Subdermal extending into muscle |

### Example 7: Osteoarthritis, Small Joints, Treatment Scheme

See Table 22 for list of locations on body where the small joint treatment protocol applies.

The minimum treatment values and maximum treatment values are based on the severity of the inflammation, mobility, and pain. Severity of the arthritis is determined by the level of inflammation, mobility, and pain symptoms. Application of therapy increases in intensity, duration and frequency as the severity increases. Minimum treatment parameters define the requirements for the initiation of the osteoarthritis care management process. Treatment includes a border around the wound site of up to 1 cm due to circulatory issues.

TABLE 21
small joint osteoarthritis treatment scheme parameters.

| Parameter | Minimum | Maximum |
|---|---|---|
| Distance from exit to site (cm) | 5 | 25 |
| Time of application (s/cm²) | 10 | 45 |
| Number of treatments | 3 | 40 |
| Length of time between treatments (hours) | 12 | 168 |
| Temperature of plasma stream at contact | 10 | 60 |
| with site (° C.) | | |
| Velocity of plasma stream at contact | 0.5 | 200 |
| with site (m/s) | | |

TABLE 22
small joint osteoarthritis body locations.

| | | |
|---|---|---|
| Ankles | Fingers | Toes |
| Elbows | Hand | Wrist |

### Example 8: Osteoarthritis, Large Joints Treatment Scheme

See Table 24 for list of locations on body where the large joint treatment protocol applies.

The minimum treatment values and maximum treatment values are based on the severity of the inflammation, mobility, and pain. Length of time is different from small joint due to the depth of the joint beneath the surface of the skin and the amount of surrounding soft tissue. Severity of the osteoarthritis is determined by the level of inflammation, mobility, and pain symptoms. Application of therapy increases in intensity, duration and frequency as the severity increases. Minimum treatment parameters define the requirements for the initiation of the osteoarthritis care management process. Treatment includes a border around the wound site of up to 1 cm due to circulatory issues.

TABLE 23
large joint osteoarthritis treatment scheme parameters.

| Parameter | Minimum | Maximum |
|---|---|---|
| Distance from exit to site (cm) | 1 | 25 |
| Time of application (s/cm²) | 15 | 120 |
| Number of treatments | 3 | 50 |
| Length of time between treatments (hours) | 12 | 168 |
| Temperature of plasma stream at contact | 10 | 60 |
| with site (° C.) | | |
| Velocity of plasma stream at contact | 0.5 | 200 |
| with site (m/s) | | |

TABLE 24
large joint osteoarthritis body location.

| | | |
|---|---|---|
| Hip | Neck | Spine |
| Knee | Shoulder | |

### Example 9: Rheumatoid Arthritis, Small Joints Treatment Scheme

The minimum treatment values and maximum treatment values are based on the severity of the inflammation, mobility, and pain. Severity of the rheumatoid arthritis is determined by the level of inflammation, mobility, and pain symptoms. Application of therapy increases in intensity, duration and frequency as the severity increases. Minimum treatment parameters define the requirements for the initiation of the rheumatoid arthritis care management process. Treatment includes a border around the wound site of up to 3 cm due to circulatory issues.

TABLE 25
small joint rheumatoid arthritis treatment scheme parameters.

| Parameter | Minimum | Maximum |
|---|---|---|
| Distance from exit to site (cm) | 3 | 25 |
| Time of application (s/cm²) | 30 | 90 |
| Number of treatments | 3 | 40 |
| Length of time between treatments (hours) | 12 | 168 |
| Temperature of plasma stream at contact with | 10 | 60 |
| site (° C.) | | |
| Velocity of plasma stream at contact with | 0.5 | 200 |
| site (m/s) | | |

TABLE 26
small joint rheumatoid arthritis body location.

Ankles
Elbows
Fingers
Hand
Toes
Wrist

### Example 10: Rheumatoid Arthritis, Large Joints Treatment Scheme

The minimum treatment values and maximum treatment values are based on the severity of the inflammation, mobility, and pain. Length of time is different from small joint due to the depth of the joint beneath the surface of the skin and the amount of surrounding soft tissue. Severity of the rheumatoid arthritis is determined by the level of inflammation, mobility, and pain symptoms. Application of therapy increases in intensity, duration and frequency as the severity increases. Minimum treatment parameters define the requirements for the initiation of the rheumatoid arthritis care management process. Treatment includes a border around the wound site of up to 1 cm due to circulatory issues.

TABLE 27
large joint rheumatoid arthritis treatment scheme parameters.

| Parameter | Minimum | Maximum |
|---|---|---|
| Distance from exit to site (cm) | 1 | 25 |
| Time of application (s/cm²) | 20 | 120 |
| Number of treatments | 3 | 60 |
| Length of time between treatments (hours) | 12 | 168 |
| Temperature of plasma stream at contact with | 10 | 60 |
| site (° C.) | | |
| Velocity of plasma stream at contact with | 0.5 | 200 |
| site (m/s) | | |

TABLE 28
large joint rheumatoid arthritis body locations.

Hip
Knee
Neck
Shoulder
Spine

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

While the present invention has been disclosed with reference to certain embodiments, numerous modifications, alterations, and changes to the described embodiments are possible without departing from the sphere and scope of the present invention, as defined in the appended claim(s). Accordingly, it is intended that the present invention not be limited to the described embodiments, but that it has the full scope defined by the language of the following claims, and equivalents thereof.

1. A method for administering a stream of gas generated by a plasma source and containing nitric oxide as the most prominent component together with other chemical and physical components to an organism, the method comprising:
creating a stream of gas that has been generated by a plasma source, in which the stream has, as part of its content, nitric oxide (NO) in a concentration from about 200 ppm to 1000 ppm at impact with dermis and directing the stream of gas at a site of action to obtain a therapeutic result by at least one of stimulating sub-dermal production of NO, mobilization of NO generated by a plasma source across and through the dermis, and the direct effect of NO in combination with other chemical and physical components of the plasma stream, including UV radiation and heat flow, on pathogens.

2. The method of claim 1, wherein directing the stream of gas at a site of action comprises positioning a device used to create the stream of gas from 1 to 25 centimeters from the device to the site.

3. The method of claim 1, wherein directing the stream of gas comprises directing the plasma-generated stream of gas with composition in claim 1 for 5 to 60 seconds for each square centimeter of site area.

4. The method of claim 1, wherein the stream of gas generated by a plasma source is directed to the site over a plurality of discrete application procedures, wherein the plurality of discrete application procedures is from 1 to 24 discrete applications.

5. The method of claim 1, further comprising directing the stream of gas to the site over multiple discrete time periods, the time periods separated by from 3 to 168 hours.

6. The method of claim 1, wherein the temperature of the stream at contact with the site is from 10 to 60 degrees C.

7. The method of claim 1, wherein the velocity of the stream at contact with the site is from 0.5 to 200 meters per second.

8. The method of claim 1, wherein the stream of gas together with other stream components is created using an electric arc discharge device.

9. The method of claim 1, wherein the stream of nitric oxide generated by a plasma source is created using a microwave discharge device.

10. The method of claim 1, wherein the stream of nitric oxide generated by a plasma source is created using a gliding arc discharge device.

11. The method of claim 1, wherein the therapeutic result is selected from the list consisting of facilitating hair-growth, reducing wrinkles, facilitating vasodilation, alleviating pain associated with osteoarthritis and Rheumatoid Arthritis, combating Gram Positive microorganisms, combating Gram Negative microorganisms, combating fungi and viruses.

12. The method of claim 1, wherein the therapeutic result is selected from the list consisting of treating osteoporosis, collagen formation, stem cell signaling, satellite cell differentiation, wound-healing, wound-management, acne, nerve regeneration, inhibiting cancer cell proliferation, promoting apoptosis, stimulating endogenous nitric oxide production, and stimulating iNOS (inducible nitric oxide synthase) pathways.

13. The method of claim 1, wherein the therapeutic result is selected from the list consisting of maintaining homeostasis in the cardiovascular and respiratory systems, causing vasodilation, easing blood pressure, cleaning the blood, reversing atherosclerosis, preventing cardiovascular diseases, slowing down atherosclerotic plaque deposition on vascular walls, suppressing bacterial infections, suppressing viruses and parasitic attacks, deterring cancer cell growth, reducing pain associated with joint swelling in arthritis, decreasing the risk of cancer, diabetes, myocardial infarction and stroke.

14. The method of claim 1, wherein the therapeutic result is selected from the list consisting of facilitating hair-growth, reducing wrinkles, facilitating vasodilation, alleviating pain associated with osteoarthritis and Rheumatoid Arthritis, combating Gram Positive microorganisms, combating Gram Negative microorganisms, combating fungi and viruses.

15. The method of claim 1, wherein directing the stream of gas generated by a plasma sources at the site of action is undertaken according to the following criteria:

| Parameter | Minimum | Maximum |
|---|---|---|
| Distance from exit to site (cm) | 1 | 25 |
| Time of application (s/cm²) | 5 | 45 |
| Number of treatments | 1 | 24 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

16. The method of claim 1, wherein directing the stream of gas generated by a plasma source at the site of action is undertaken according to the following criteria:

| Parameter | Minimum | Maximum |
|---|---|---|
| Distance from exit to site (cm) | 1 | 25 |
| Time of application (s/cm²) | 10 | 90 |
| Number of treatments | 1 | 24 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

17. The method of claim 1, wherein directing the stream of gas generated by a plasma source at the site of action is undertaken according to the following criteria:

| Parameter | Minimum | Maximum |
|---|---|---|
| Distance from exit to site (cm) | 1 | 25 |
| Time of application (s/cm²) | 1 | 45 |
| Number of treatments | 1 | 200 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

18. The method of claim 1, wherein directing the stream of gas generated by plasma sources at the site of action is undertaken according to the following criteria:

| Parameter | Minimum | Maximum |
|---|---|---|
| Distance from exit to site (cm) | 1 | 25 |
| Time of application (s/cm²) | 5 | 90 |
| Number of treatments | 1 | 200 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

19. The method of claim 1, wherein directing the stream of gas generated by plasma sources at the site of action is undertaken according to the following criteria:

| Parameter | Minimum | Maximum |
|---|---|---|
| Distance from exit to site (cm) | 1 | 25 |
| Time of application (s/cm²) | 5 | 45 |
| Number of treatments | 1 | 24 |
| Length of time between treatments (hours) | 3 | 168 |
| Temperature of plasma stream at contact with | 10 | 60 |
| site (° C.) | | |
| Velocity of plasma stream at contact with | 0.5 | 200 |
| site (m/s) | | |

20. The method of claim 1, wherein directing the stream of gas generated by plasma sources at the site of action is undertaken according to the following criteria:

| Parameter | Minimum | Maximum |
|---|---|---|
| Distance from exit to site (cm) | 10 | 30 |
| Time of application (s/cm²) | 10 | 90 |
| Number of treatments | 1 | 200 |
| Length of time between treatments (hours) | 1 | 168 |
| Temperature of plasma stream at contact with | 10 | 50 |
| site (° C.) | | |
| Velocity of plasma stream at contact with | 0.5 | 200 |
| site (m/s) | | |

21. The method of claim 1, wherein directing the discrete stream of matter at the site of action is undertaken according to the following criteria:

| Parameter | Minimum | Maximum |
|---|---|---|
| Distance from exit to site (cm) | 5 | 25 |
| Time of application (s/cm²) | 10 | 45 |
| Number of treatments | 3 | 40 |
| Length of time between treatments (hours) | 12 | 168 |
| Temperature of plasma stream at contact with | 10 | 60 |
| site (° C.) | | |
| Velocity of plasma stream at contact with | 0.5 | 200 |
| site (m/s) | | |

22. The method of claim 1, wherein directing the discrete stream of matter at the site of action is undertaken according to the following criteria:

| Parameter | Minimum | Maximum |
|---|---|---|
| Distance from exit to site (cm) | 1 | 25 |
| Time of application (s/cm²) | 15 | 120 |
| Number of treatments | 3 | 50 |
| Length of time between treatments (hours) | 12 | 168 |
| Temperature of plasma stream at contact with | 10 | 60 |
| site (° C.) | | |
| Velocity of plasma stream at contact with | 0.5 | 200 |
| site (m/s) | | |

23. The method of claim 1, wherein directing the discrete stream of matter at the site of action is undertaken according to the following criteria:

| Parameter | Minimum | Maximum |
|---|---|---|
| Distance from exit to site (cm) | 3 | 25 |
| Time of application (s/cm²) | 30 | 90 |
| Number of treatments | 3 | 40 |
| Length of time between treatments (hours) | 12 | 168 |
| Temperature of plasma stream at contact with | 10 | 60 |
| site (° C.) | | |
| Velocity of plasma stream at contact with | 0.5 | 200 |
| site (m/s) | | |

24. The method of claim 1, wherein directing the stream of gas generated by a plasma sources at the site of action is undertaken according to the following criteria:

| Parameter | Minimum | Maximum |
|---|---|---|
| Distance from exit to site (cm) | 1 | 25 |
| Time of application (s/cm²) | 20 | 120 |
| Number of treatments | 3 | 60 |
| Length of time between treatments (hours) | 12 | 168 |
| Temperature of plasma stream at contact with | 10 | 60 |
| site (° C.) | | |
| Velocity of plasma stream at contact with | 0.5 | 200 |
| site (m/s) | | |
